(19) United States
(12) Patent Application Publication
(10) Pub. No.: US 2006/0223197 A1
(43) Pub. Date: Oct. 5, 2006

(54) Method and apparatus for the detection of biological molecules

(76) Inventor: Claus Vielsack, Birkenfeld (DE)

Correspondence Address: INTELLECTUAL PROPERTY / TECHNOLOGY LAW, PO BOX 14329, RESEARCH TRIANGLE PARK, NC, 27709, US

(21) Appl. No.: 11/396,454

(22) Filed: Apr. 3, 2006

(30) Foreign Application Priority Data
Apr. 5, 2005 (GB) 0506880.4

(51) Int. Cl. G01N 33/551

(52) U.S. Cl. 436/524; 977/900

(57) ABSTRACT

A detection system and a method for the detection of a plurality of substances is disclosed. The detection system has a plurality of detection probes, each of the detection probes having an up-conversion fluorescing core of dimensions less than 200 nm and is linked to an affinity moiety. The affinity moiety bonds to one of the plurality of substances.

### CROSS-REFERENCE TO RELATED APPLICATION

This application claims priority under 35 USC 119 of United Kingdom Patent Application No. 0506880 filed Apr. 5, 2005.

### FIELD OF THE INVENTION

The invention relates to a method and an apparatus for the detection of biological molecules.

### PRIOR ART

Traditional methods for the detection of biological molecules—also called biomolecules—in vivo and in vitro rely on the use of radioactive markers as labels. These labels are effective because of the high degree of sensitivity for the detection of radioactivity. However, there are difficulties with using radioisotopes as radioactive markers. These difficulties include the need to train personnel in their use, as well as the general safety issues associated with the use of radioisotopes. Furthermore many radioisotopes have inherently short half-lives.

As a result current efforts have shifted towards the utilisation of chemofluorescent molecules as tags. Fluorescence is the emission of light resulting from the absorption of radiation at one wavelength (excitation) followed by nearly immediate radiation at a different wavelength (emission). Chemofluorescence methods have the disadvantage of photobleaching, low fluorescence intensity, short half-lives, broad spectral line widths and non-gaussian asymmetric emission spectra having long tails.

Another solution for the detection of biological molecules is known from U.S. Pat. No. 6,326,144 (Bawendi et al, assigned to MIT). This patent document teaches a composition comprising fluorescent semiconductor nanocrystals for the detection of biological molecules. In operation the composition is introduced into an environment containing a biological target and the fluorescent nanocrystal composition associates with the biological target. The composition:target complex may be spectroscopically viewed by irradiating the composition:target complex with an excitation light source. The fluorescent nanocrystal composition emits a characteristic emission spectrum which can be observed and measured spectrophotometrically.

Unfortunately such semiconductor nanocrystals described in this patent application are limited in their application. They show increasing photoluminescence intensity under continuous excitation which increases towards a maximum value. This is due to the presence of traps in the nanocrystals which are gradually saturated. This can affect quantitative measurements. The size of the nanocrystals at 3-10 nm is comparative to the size of some of the biological molecules, such as proteins, to which they bound and, as a results, the biological functionalist of the process under study can be affected.

A further disadvantage to the use of such molecules is thermoquenching in which the luminescence of the semiconductor nanocrystal increases with increasing temperature. Whilst this is not a problem when the experiments are performed at room temperature, it can cause difficulties when experiments are performed at elevated temperatures, such as during PCR.

It is particularly useful to be able to label several biological molecules in the same experiment so that the interaction between more than one biological molecule can be observed. This is termed multiplexing. Multiplexing is almost impossible to achieve using organic dyes since the discrete excitation energies of the individual dyes make the excitation with a single light source impossible. Furthermore, the long red tail of the emission characteristic makes the differentiation between various dyes more or less impossible.

Another problem that exists in prior art systems is the autofluorescence of most proteins and other ones of the biological molecules. Unless this “background” fluorescence is eliminated from the results, it may cause problems in interpreting the results.

The use of up-conversion fluorescing materials for the detection of cell and tissue surface antigens has been discussed in an article by Auzel “Up-conversion and Anti-Stokes Processes with f and d ions in solids”, Chem. Rev. 2004, 104, 139-172 (see in particular page 169). Auzel points out that the use of IR-up-converting phosphors is that they cannot excite the natural biological materials and thus provide a good detection contrast with respect to autofluorescence than prior art systems.

A practical method for the use of up converting phosphors for the detection of antigens is taught in “Detection of Cell and Tissue Surface Antigens using up-converting phosphors: a new reporter technology” by Zijlmans et al, Analytical Biochemistry, 267, 30-36 (1999). The up-converting phosphors created using the methods disclosed in this article have a dimensions in the region of 0.2-0.4 μm. Such large particles have the disadvantage that they can interact themselves with an analyte and thus reduce the sensitivity and the specifity of the detection system.

### SUMMARY OF THE INVENTION

It is therefore an object of the invention to provide a system for the detection of a plurality of biological molecules.

These and other objects of the invention are solved by providing a detection system for the detection of a plurality of substances comprising a plurality of detection probes, each of the detection probes having an up-conversion fluorescing core of dimensions less than 200 nm linked to an affinity moiety. The affinity moiety bonds to one of the plurality of substances. The detection system allows the experimenter to detect a number of different substances, which are preferably biological molecules, since a number of different probes are present. Each one of the probes has a different up-conversion fluorescing core and a different affinity moiety. As a result the experimenter can detect the presence or absence of a particular substance by examining the emission spectra. The use of an up-conversion fluorescing core eliminates the autofluorescence of any of the biological molecules.

In a preferred embodiment of the invention, the up-conversion fluorescing core is surrounded by a shell, preferably made of functionalised silica. This shell allows the attachment of the biological molecule either directly to the external surface of the shell or by means of a linker. Functional groups used include, but are not limited to, thiol groups.

One example of a detection system of the invention has further probes attached to a surface of, for example, a microplate. The substances first bind to the further probes through affinity moieties and then the detection probes bind to the substances.

The further probes can also be bound to beads, such as magnetic beads, which allow the separation of the detected substances using a property of the beads. For example a magnetic field might be applied to the detection system and if magnetic beads are used on the further probes, then these will be attracted by the magnetic pole. Alternatively the beads may be comparatively large and separation could be carried out in a flow channel.

In a preferred embodiment of the invention, the up-conversion fluorescing core is made from a doped sodium yttrium fluoride. Other compositions could be used.

The object is also solved by a method for the detection of a plurality of substances which comprises:
a first step of the provision of a plurality of detection probes having an up-conversion fluorescing core linked to an affinity moiety;
a second step of placing the plurality of detection probes in contact with a fluid including the one or more substances;
a third step of exposing the probes to light of a first energy; and
a fourth step of detecting light of a second energy emitted from the one or more of the probes.

There are at least two detection probes used in order to allow multiplexing.

The method also includes in one embodiment a washing step to wash away the fluid. Thus any unbound substances are removed from the detection system. This reduces the risk of erroneous results due to emission spectra from other substances unrelated to the substances of interest.

### DESCRIPTION OF THE DRAWINGS

FIG. 1 shows a detector probe according to the invention.

FIG. 2 shows an example of the invention in an immunoassay.

FIG. 3 is a flow diagram to illustrate the method of the invention.

FIG. 4 shows a further example of the invention in a fluid.

### DETAILED DESCRIPTION OF THE INVENTION

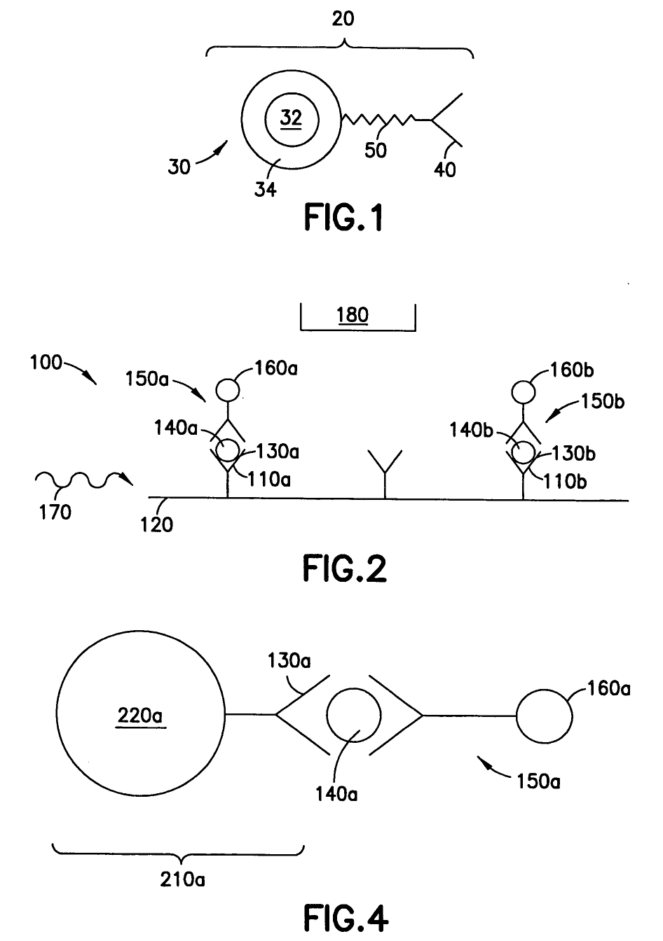

FIG. 1 shows the principle of operation of the method of detection of a biological molecule 10. The biological molecules include, but are not limited to, proteins, nucleic acids, cells, subcellular organelles. A probe 20 comprises a nanoparticle 30 to which an affinity moiety 40 is connected by means of a linker 50. The affinity moiety 40 is the moiety which couples with the biological molecule 10 being detected. The affinity moiety 40 could, for example, be an anti-body or other ligand. The nanoparticle 30 comprises an up-conversion fluorescing core 32 surrounded by a shell 34. The shell 34 is preferably a silica shell but could be another polymer or inorganic network.

A nanoparticle in the context of this invention is a particle with a maximum dimension of 200 nm.

The up-conversion fluorescing core 32 can be made by a number of methods. These include creating a micro-sized particle and then grinding the particle down to form a particle with dimension in the nanometre range. Another method is to synthesize the nanoparticle.

Yi et al in the paper “Synthesis, Characterization, and Biological Application of Size-Controlled Nanocrystalline NaYF4:Yb,Er Infrared-to-Visible Up-Conversion Phosphors” published in Nano Letters, vol 4, no 11, 2191-2196, 2004, describe such a method. The method uses stock solutions of yttrium oxide, ytterbium oxide and erbium oxide and dissolving it into hydrochloric acid at an elevated temperature and adjusted to pH 2.

NaYF4:Yb,Er nanoparticles for the fluorescing core 32 were prepared by dissolving NaF in deionised water. Another solution was prepared by mixing together 16 mL of 0.2 M YCl3, 3.4 mL of 0.2 M YbCl3, 0.6 mL of 0.2 M ErC13 and 20 mL of 0.2 M EDTA stock solutions to form the metal-EDTA complex. The complex solution was injected into the NaF solution quickly, and the mixture was stirred for 1 h at room temperature. Precipitates from the reaction were centrifuged, washed three times using deionised water and once with anhydrous ethanol. The precipitates of NaYF4:Yb,Er nanoparticles were then dried under vacuum, and a white powder obtained.

Annealing of the nanoparticles was carried out under a hydrogen/nitrogen atmosphere by heating them to the desired temperature at a rate of 20° C. per minute, and maintaining this temperature for 5 hours. After annealing, the nanoparticles were cooled down naturally to room temperature under the same atmosphere. The fluorescing core 32 was covered with the shell 34 from silica by the hydrolysis of tetraethyl orthosilicate.

Different types of nanoparticles with different emission spectra can be produced by creating nanoparticles of different sizes. Since the peaks of the emission spectra are quite sharp (between approx. 30 and 50 nm in the wavelength domain), even quite small differences in sizes can lead to emission spectra which are different from each other and thus sufficiently different to be detectable.

Having formed the shell 34 from silica, the surface is functionalised (for example with a thiol group) and the linker 50 attached to the surface. The affinity moiety 40 is attached to the linker 50. Examples of such linkers 50 include but are not limited to functionalised fatty acids and aliphatic linkers.

Use of the probe 20 is now shown with respect to FIG. 2 which could be an immunoassay such as a sandwich assay. FIG. 2 shows an assay 100 with a number of surface probes 110a, b immobilised on the surface of a plate 120. The plate 120 could be, for example, made of polystyrene. The surface probes 110a and 110b have affinity moieties 130a and 130b attached to them which attracts biological molecules 140a and 140b. The affinity moieties 130a and 130b are different in nature and therefore attract different biological molecules 140a and 140b. Detection probes 150a and 150b have fluorescing nanoparticles 160a and 160b attached to them. The fluorescing nanoparticles 160a and 160b fluoresce at different wavelengths and are thus distinguishable from each other. The detection probes 150a and 150b are attracted to different ones of the biological molecules 140a and 140b as shown in FIG. 2. An investigator therefore needs to shine light of a desired wavelength onto the assay 100 and measure the excitation spectra to determine which ones of the detection probes 150a and/or 150b are present on the assay 100 in order to work out which ones of the biological molecules 140a and 140b are present.

The light can be either shone down on the assay 100 from above. In this case the assay 100 must be washed prior to ensure that no unbound probes 150a and 150b remain on the surface of the assay 100 and thus confuse the measurements. Alternatively, light can be shone along the surface of the assay 100 as indicated by an arrow 170. The light propagates substantially along the surface of the plate 120 and only excites the fluorescing particles 160a and 160b bound to the surface probes 110a and 110b. A detector 180 detects and records the emission spectra. In one embodiment of the invention the detector incorporates an acoustic-optical transfer filter.

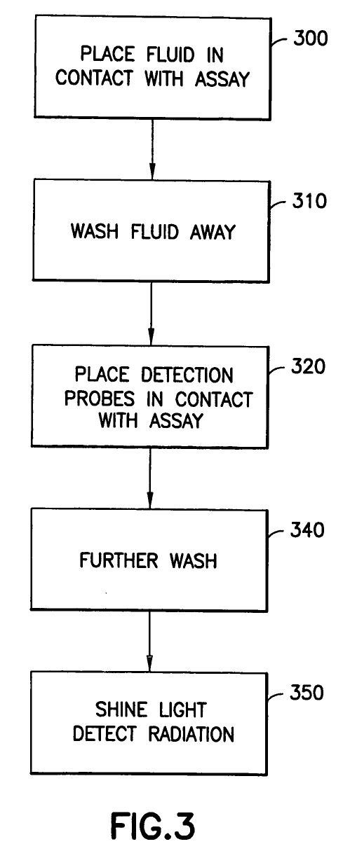

An experiment to detect biological molecules 140a and 140b in a fluid will now be described with respect to FIG. 3. In a first step 300 the experimental fluid will be placed in contact with the surface of the assay 100. The biological molecules 140a and 140b come into contact with the affinity moieties 130a and 130b and a number of biological molecules 140a and 140b become covalently bound to the surface probes 110a and 110b. The experimental fluid is then washed away in step 310 and a probe solution containing the detection probes 150a and 150b is placed into contact with the assay 100 in step 320. Those detection probes 150a and 150b which can bind to the biological molecules 140a and 140b are bound to the surface probes 110a and 110b, for example by a covalent bond or a biotin-streptavidin system. Any further complementary probes 150a, 150b with no corresponding biological molecules 140a, 140b will not become bound to the surface probes 110a and 110b and can be washed away in step 340 (if wished). Light is then shone upon the surface of the assay 100 as described above and the presence or absence of biological molecules 140a and 140b determined based upon the fluorescence spectra emitted is detected in step 350.

In another embodiment of the experiment shown in FIG. 4, the probe 210a is not bound to the surface of the plate 120 (as in FIG. 2) but have a polystyrene bead or magnetic particle 220a attached to it as shown in FIG. 4. The rest of the features of FIG. 4 are the same as those shown in FIG. 2 and the same reference signs are used. The probe 210a with the bound biological molecule 140a can be separated from the unbound biological molecules by the use of a magnetic field and/or a flow device.

The above examples are described with respect to two individual biological molecules 140a and 140b. The principles are, however, applicable to a large number of biological molecules 140a and 140b. It is only necessary for sufficient number of differing fluorescing nanoparticles 160a and 160b to be available to allow clear identification of the biological molecules 140a and 140b from the analysis of the emission spectra.

The foregoing is considered illustrative of the principles of the invention and since numerous modifications will occur to those skilled in the art, it is not intended to limit the invention to the exact construction and operation described. All suitable modifications and equivalents fall within the scope of the claims.

1. A detection system for the detection of a plurality of substances comprising:
a plurality of detection probes, each of the detection probes having an up-conversion fluorescing core of dimensions less than 200 nm linked to an affinity moiety, the affinity moiety bonding to one of the plurality of substances.

2. The detection system of claim 1 further having a light source for exciting the up-conversion fluorescing core.

3. The detection system of claim 1 further having at least one detector for measuring the radiation emitted from the up-conversion fluorescing core.

4. The detection system of claim 1 wherein the up-conversion fluorescing core is surrounded by a shell.

5. The detection system of claim 4, wherein the shell is a functionalised silica shell.

6. The detection system of claim 1 comprising a first linker between the fluorescing core and the affinity moiety.

7. The detection system of claim 1 further comprising a further probe having a further affinity moiety bonding to one of the plurality of substances.

8. The detection system of claim 7 wherein the further probe is attached to a surface.

9. The detection system of claim 7, wherein the further probe is attached to a bead.

10. The detection system of claim 1, wherein the up-conversion fluorescing core is made from a doped sodium yttrium fluoride or another rare-earth element containing compound.

11. A method for the detection of a plurality of substances comprising
a first step of the provision of a plurality of detection probes having an up-conversion fluorescing core linked to an affinity moiety;
a second step of placing the plurality of detection probes in contact with a fluid including the one or more substances;
a third step of exposing the plurality of detection probes to light of a first energy; and
a fourth step of detecting light of a second energy emitted from the one or more of the plurality of detector probes.

12. The method of claim 11, wherein there are at least two ones of the plurality of detection probes with different affinity moieties.

13. The method of claim 11, wherein the fifth step of detecting light of a second energy is carried out using a photodetector having an acoustic-optical transfer filter.

14. The method of claim 11, further including at least one washing step to wash away the fluid.

15. The method of claim 11, wherein the second step is preceded by a step of placing the fluid in contact with a plurality of further probes, the further probes having a further affinity moiety for binding with one of the plurality of substances.

16. The method of claim 15, wherein the further probes are attached to a bead and the detector probes are separated within the fluid using a property of the bead.
